(19) United States
(12) Patent Application Publication
(10) Pub. No.: US 2006/0275887 A1
(43) Pub. Date: Dec. 7, 2006

(54) Mycobacteria compositions and methods of use in bioremediation

(75) Inventors: Charles D. Miller, North Logan, UT (US); Anne J. Anderson, Providence, UT (US); Ronald C. Sims, Logan, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: Utah State University, Logan, UT (US)

(21) Appl. No.: 11/446,274

(22) Filed: Jun. 2, 2006

Related U.S. Application Data
(60) Provisional application No. 60/687,567, filed on Jun. 3, 2005.
(60) Provisional application No. 60/693,452, filed on Jun. 23, 2005.

(51) Int. Cl. C12N 1/21

(52) U.S. Cl. 435/262.5; 435/252.3

(57) ABSTRACT

The present invention includes a contaminant-degrading composition for use in remediation of contaminated soil having a selected contaminant. Such a composition can include a seed for a plant capable of growing in the presence of the selected contaminant, and a contaminant-degrading mycobacteria on the seed. Additionally, the present invention includes a contaminant-degrading system for use in remediation of contaminated soil having a selected contaminant. Such a system can include a plant growing in the contaminated soil, and contaminant-degrading mycobacteria colonized on a root of the plant, wherein the mycobacteria is capable of degrading the selected contaminant. The mycobacteria can be capable of degrading the selected contaminant, such as PAHs, PCPs, MTBEs, and the like. Additionally, the contaminant-degrading mycobacteria can be at least one of M. KMS, M. JLS, or M MCS. Also, the contaminant-degrading mycobacteria can have nid dioxygenase genes, which can further include a nidB-nidA sequence motif.

### CROSS-REFERENCE TO RELATED APPLICATIONS

This United States patent application claims benefit of U.S. Provisional Patent Application Ser. No. 60/687,567, entitled “IDENTIFYING AND PROPAGATING POLYCYCLIC AROMATIC HYDROCARBON-DEGRADING MYCOBACTERIA,” filed on Jun. 3, 2005, with Charles D. Miller, Anne J. Anderson, and Ronald C. Sims as inventors, and also claims benefit of U.S. Provisional Patent Application Ser. No. 60/693,452, entitled “PROBES AND METHODS FOR IDENTIFYING POLYCYCLIC AROMATIC HYDROCARBON-DEGRADING MYCOBACTERIA,” filed Jun. 23, 2005, with Charles D. Miller, Anne J. Anderson, and Ronald C. Sims as inventors, which are incorporated herein by reference. This United States patent application cross-references United States patent application having Attorney Docket No. 14185.7.3.1, entitled “PROBES AND METHODS FOR IDENTIFYING POLYCYCLIC AROMATIC HYDROCARBON-DEGRADING MYCOBACTERIA,” filed concurrently herewith, which is incorporated herein by reference.

The U.S. Government has a paid-up license in this invention and the right in limited circumstances to require the patent owner to license others on reasonable terms as provided for by the terms of Grant No. A08379 awarded by the National Science Foundation.

### BACKGROUND OF THE INVENTION

1. The Field of the Invention

The present invention relates to compositions having mycobacteria capable of degrading contaminants in soil. More particularly, the present invention relates to methods of using such mycobacterial compositions to degrade contaminants in soil by including the mycobacterial composition with seeds and/or with plant roots.

2. The Related Technology

Many industries use and/or generate toxic chemicals in systems, equipment, and processes during the production of the vast array of commercial products on the market even though the products themselves may or may not present toxic characteristics. As a consequence, the soil and environment near or downstream from industrial sites often becomes contaminated. While various remediation techniques have been developed to decontaminate soil, various complex organic compounds are difficult to remove or break down. Examples of noxious soil contaminants include the organic compounds known as polycyclic aromatic hydrocarbons (“PAH”), polychlorinated phenols (“PCP”), and methyl tertiary butyl ether (“MTBE”), which are commonly present in soil around industrial sites and have toxic, mutagenic, and carcinogenic properties.

Various types of soil remediation techniques have been developed in order to remove PAHS, PCPS, MTBES, and other contaminants from the areas surrounding abandoned industrial sites. Bioremediation is one remediation technique that uses living organisms (e.g., bacteria) to clean up oil spills or remove other pollutants, such as PAHs, PCPs, MTBEs, and other contaminants, from soil, water, and wastewater. Bioremediation of soils has been shown to be a promising technique when microorganisms were determined to be capable of naturally degrading the contaminating chemicals. However, bioremediation may not be a suitable technique when contaminant-degrading microorganisms are not available for degrading a particular chemical or class of chemicals (e.g., PAH, PCP, MTBE) present in a site needing decontamination.

Therefore, it would be advantageous to have a composition containing microorganisms that are capable of degrading various soil contaminants such as low molecular weight and/or high molecular weight PAHs, PCPs, MTBEs, and the like. Additionally, it would be beneficial to be capable of inoculating contaminated soil with contaminant-degrading microorganisms so that the microorganisms can be used for bioremediation. More particularly, it would be beneficial to treat contaminated soil by planting seeds or seedlings such that the contaminant-degrading mycobacteria can grow on or proximal to the plant roots to enhance bioremediation and/or phytoremediation.

### SUMMARY OF THE INVENTION

Generally, the foregoing deficiencies in the art can be solved by embodiments of the present invention, which can be employed to use microorganisms that are capable of degrading various soil contaminants such as low molecular weight and/or high molecular weight PAHs. Additionally, embodiments of the present invention can include compositions having a contaminant-degrading microorganism that can be applied to contaminated soil. Further embodiments can include the use of contaminant-degrading microorganisms as they colonize the roots of plants growing in contaminated soil so that the microorganisms can be used for bioremediation and/or in phytobioremediation.

In one embodiment, the present invention includes a contaminant-degrading composition for use in remediation of contaminated soil having a selected contaminant. Such a composition can include a seed for a plant capable of growing in the presence of the selected contaminant, and a contaminant-degrading mycobacterium on the seed, wherein the mycobacteria is capable of degrading the selected contaminant.

In one embodiment, the present invention includes a contaminant-degrading system for use in remediation of contaminated soil having a selected contaminant. Such a system can include a plant growing in the contaminated soil, and contaminant-degrading mycobacteria colonized on a root of the plant, wherein the mycobacteria is capable of degrading the selected contaminant.

The mycobacteria can be capable of degrading the selected contaminant, such as PAHs, PCPs, MTBEs, and the like. Additionally, the contaminant-degrading mycobacteria can be at least one of M. KMS, M. JLS, or M. MCS. Also, the contaminant-degrading mycobacteria can have a nid dioxygenase gene. Further, the contaminant-degrading mycobacteria can include a nidB-nidA sequence motif. Furthermore, the contaminant-degrading mycobacteria can include selected gene sequences that identify the capability of degrading selected contaminants, such as selected gene sequences that identify the capability of degrading PCPs, MTBEs, or other similar selected contaminants.

Additionally, while any plant can be used, it is preferable for the plant to be capable of growing and thriving in contaminated soil so that the plant is substantially healthy and capable of substantially normal function. Examples of such plants include barley, wheatgrass, Lolium species, legumes, alfalfa, rice, grasses, forbs, trees, mulberry tree, clover, corn, brassicas, curcurbits, and rye.

In one embodiment, the mycobacteria can be provided or added to a seed while in a composition including substances useful for growing and propagating mycobacteria. Examples of such substances include root wash, root extract, D-mannitol, D-psicose, propionic acid, D-sorbitol, sucrose, alpha-cyclodextrin, and sedoheptulosan, polyoxyethylene sorbitan mono-palmitate (Tween 40), polyoxyethylene sorbitan monooleate (Tween 80), D-fructose, D-mannose, D-trehalose, or pyruvic acid methyl ester. Additionally, beneficial substances can include complex mixtures containing polysaccharides and other nutrients (e.g., molasses, whey effluent, and the like).

In one embodiment, the present invention includes a method of decontaminating soil having a selected contaminant. Such a method can include growing a plant in contaminated soil having a selected contaminant such that contaminant-degrading mycobacteria colonizes the roots of the plant. This can include placing the plant and/or the mycobacteria in the soil in order to colonize the contaminant-degrading mycobacteria on the root of the plant. Additionally, the method can include planting a seed in the soil, said seed for a plant capable of growing in the presence of the selected contaminant. This can further include applying the contaminant-degrading mycobacteria to the seed. In some instances the seed would be treated with the contaminant-degrading mycobacteria before being planted. In other instances the contaminant-degrading mycobacteria is applied to soil adjacent to at least one of the seed after planting or the plant.

Additionally, the method can include a process of applying a composition having the contaminant-degrading mycobacteria to the soil. This can include applying the composition in any form from solid to liquid. For example, a liquid composition can be sprayed in the soil in an effective amount so that the mycobacteria are able to be associated with the seed and/or colonize the root, or be applied as pellets or in a fertilizer. Also, the seed can be dipped in a liquid composition so that the seed includes the liquid containing the contaminant-degrading mycobacteria at the time of planting. Moreover, the mycobacteria inoculum can be added to roots of established plants in order to facilitate colonization of the roots.

One embodiment of the present invention is a method for determining whether a microorganism is a PAH-degrading mycobacteria. Such a method includes: providing a first set of DNA molecules consisting of fragments of genomic DNA of at least one mycobacteria species capable of biodegrading a PAH; contacting, under hybridizing conditions, the first set of DNA molecules with a second set of DNA molecules consisting of genomic DNA of an unknown mycobacteria species isolated from a sample; and detecting hybridization between the first set of DNA molecules and the second set of DNA molecules, wherein the hybridization between the first and second sets is an indication that the unknown mycobacteria species is a PAH-degrading mycobacteria.

One embodiment of the present invention is a method of identifying the presence of a PAH-degrading mycobacteria having a nidB-nidA sequence motif in dioxygenase genes in a soil sample. Such a method includes: providing at least one primer set capable of hybridizing with a nid dioxygenase nucleotide sequence, such as a nidB-nidA sequence motif; hybridizing the at least one primer with the nid dioxygenase nucleotide sequence; producing a polymerase chain reaction (“PCR”) product; and determining whether the PCR product indicates the presence of a PAH-degrading mycobacteria, which can include size migration on an electrophoretic gel.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIGS. 1A-1E are embodiments of seeds having a mycobacteria thereon;

FIG. 2 is a graph illustrating the ability of mycobacteria to form a biofilm;

FIG. 3 is a graph illustrating the ability of mycobacteria to have planktonic growth;

FIG. 4 is a graph illustrating a mycobacterial colony forming units on roots when plants are grown in a microbially-contaminated soil mix;

FIGS. 5A-5B are photographs illustrating mycobacterial colonies on roots growing on plate medium from roots of seedlings grown from inoculated barley seeds;

FIGS. 6A-6D are graphs illustrating a mycobacterial colony forming units on root sections along the length of the root;

FIG. 7 is a graph illustrating pyrene mineralization in microcosms containing barley with and without root colonization with a mycobacterium or with just microbial amendment of the growth medium;

FIG. 8 is a table showing the mass balance for recovery of label from radioactive pyrene from the microcosms described in FIG. 7;

FIG. 9 is a graph illustrating MTBE mineralization;

FIG. 10 is a graph illustrating TBA mineralization; and

FIG. 11 is a graph illustrating MTBE and TBA mineralization.

### DETAILED DESCRIPTION OF THE PREFERRED EMBODIMENTS

Generally, embodiments of the present invention are related to compositions having contaminant-degrading mycobacteria and methods of using such compositions in remediation to decontaminate soil contaminated with PAHs, PCPs, MTBEs, and other like contaminants. Also, the compositions can be combined with seeds or plant roots in order to enhance bioremediation. In part, this is because the mycobacteria can use the exudates from the roots of the plant produced by the seed or the roots of an established plant as a substrate for growth, and propagation of the contaminant-degrading mycobacteria around the plant. The contaminant-degrading mycobacteria can be identified by DNA nucleotide sequences indicative of such microorganisms that are provided in the incorporated references. These DNA sequences, or portions thereof, can be used as probes in order to determine whether the soil, roots, or the like contain microorganisms with a nidB-nidA sequence in genes that encode for nid dioxygenase enzymes.

I. Introduction

Nid dioxygenase genes, especially those having the nidB-nidA motifs, have been shown by the inventors to be present in microorganisms that can biodegrade PAHs, PCPs, MTBEs, and/or other like contaminants. Assays that can identify the presence of nid dioxygenase genes in various types of samples can be valuable for finding new contaminant-biodegrading microorganisms and determining whether or not contaminated soils contain such microorganisms. As such, microorganisms having the nidB-nidA motifs can be identified by methods and assays that do not rely on time-consuming and tedious processes that require culturing microorganisms on contaminated mediums (e.g., PAH-contaminated media), which can take days and are fraught with uncertainty. After identification of microorganisms having the nidB-nidA motifs are identified, such microorganisms can be cultured in an appropriate media and/or applied to contaminated soil.

One embodiment of the present invention is a method for determining whether a microorganism is capable of biodegrading by assaying for the presence of nidB-nidA dioxygenase DNA sequences in the genome. Typically, such a method is performed when there is not any indication the microorganism has the ability to degrade a selected contaminant or group of contaminants. A microorganism can be shown to be capable of degrading a selected contaminant or group of contaminants by having the ability to grow in a medium that includes the presence of the selected contaminant or group of contaminants. In part, this is because a selected contaminant or group of contaminants is known to be toxic to most living organisms, and the ability to grow and replicate in a contaminated environment indicates contaminant-biodegradability.

Previously, the inventors showed that PAH-biodegrading microorganisms can be found in PAH-contaminated soils. The microorganisms, such as Mycobacterium JLS (“JLS”), Mycobacterium KMS (“KMS”), and Mycobacterium MCS (“MCS”) isolates, where shown to be capable of biodegrading PAHs by being cultured on a medium in the presence of a PAH such as pyrene. Additionally, the inventors showed further PAH-biodegradation capabilities by these isolates utilizing phenathrene and benzo[a]pyrene. Further, the JLS, KMS, and/or MCS mycobacteria capable of degrading PAHs have also been shown to be capable of degrading other contaminants, such as MTBEs. Also, it has been found that the JLS, KMS, and/or MCS mycobacteria include gene sequences indicative of a capability of degrading other contaminants such as PCPs. Thus, it is contemplated that the JLS, KMS, and/or MCS mycobacteria can be used for bioremediation of sites contaminated with PAHs, PCPs, MTBEs, and other like contaminants.

Additionally, the inventors showed that the PAH-biodegrading microorganisms can be identified by analysis of their fatty acid content and sequence of their 16S ribosomal genes. As such, MIDI (Newark, Del.) performed analysis of the fatty acid content as described in the incorporated references. Also, the 16S ribosomal genes were assayed by PCR analysis with primers identified in the Sequence Listing of the incorporated references. The fatty acid content and 16S ribosomal gene analysis provided a phylogenic indication that the microorganisms were mycobacterium. The phylogenic analysis indicated the PAH-biodegrading organisms to be mycobacterium isolates. The JLS, KMS, and MCS taxonomic relation to other mycobacterium provides a basis for some potential similarities with other mycobacteria, some of which also have PAH-biodegrading capabilities. Additional information regarding the phylogenic analysis and other PAH-biodegrading mycobacteria can be found in the incorporated references.

The foregoing illustrates that contaminant-biodegrading microorganisms can be isolated from soils by being cultured on contaminated mediums. While the foregoing experimental techniques can be employed to find and identify contaminant-biodegrading microorganisms for use in biorememdiation, one embodiment of the present invention provides an improvement for identifying the presence of such contaminant-degrading mycobacterium by isolating DNA directly from a soil sample and amplifying nid dioxygenase genes (e.g., nidB-nid-A).

A. Bioremediation

Bioremediation of contaminated soils can be performed with microorganisms, such as the mycobacteria strains JLS, KMS, and MCS, that are capable of degrading contaminants, such as PAHs, PCPs, MTBEs, and other like contaminants. The mycobacteria strains JLS, KMS, and MCS have been characterized to have certain nidB-nidA sequence motifs, which appear to be indicators of the nid dioxygenase enzyme that is useful in degrading contaminants, such as PAHs and other like contaminants. After microorganisms having the nidB-nidA sequence motifs are identified, such microorganisms can be cultured in an appropriate media and/or applied to contaminated soil for bioremediation. Optionally, such microorganisms can be used with plants in an enhanced process of phytoremediation.

B. Phytoremediation

Generally, phytoremediation involves the use of plants to clean up sites that have been contaminated with chemicals or petroleum products. As such, the plants can be used to remove hazardous substances from the soil. Generally, the plants absorb contaminated water through their roots, and retain the contaminant within themselves or process the contaminants into harmless substances. Some plants can remediate soils and/or water to eliminate or decrease contamination by the uptake (e.g., transpiration) of contaminated water or contaminants from the soil. The plants can then be used to contain, remove, and/or degrade the absorbed contaminants. Phytoremediation is a cost-effective method for on-site clean-up, and is well suited for large surface areas such as those designated as “brownfields” within urban settings or sites where soil excavation and removal is difficult. While phytoremediation can be a viable option for removal or degradation of some contaminants, it can be a slow process that may or may not completely breakdown the contaminants into harmless substances. Thus, it may be beneficial to supplement phytoremediation efforts with bioremediation by inoculating the soil around the plants with microorganisms, such as the mycobacterial strains JLS, KMS, and MCS, that are capable of degrading contaminants, such as PAHs, MTBEs, and other like contaminants. The combination of phytoremediation and bioremediation is referred to herein as “phytobioremediation.” Accordingly, the term “phytobioremediation” is meant to include a combination of both plant-based phytoremediation and microorganism-based bioremediation.

C. Phytobioremediation

In one embodiment of the present invention, phytobioremediation of soils contaminated with PAHs, PCPs, MTBEs, and other like contaminants can be performed by including contaminant-degrading microorganisms on or proximal to the roots of a plant. More particularly, phytobioremediation can be performed by applying microorganisms, such as the mycobacteria strains JLS, KMS, and MCS, that are capable of degrading contaminants, such as PAHs, PCPs, MTBEs, and other like contaminants, to the root of plants. Accordingly, phytobioremediation can be conducted by the following: applying mycobacteria to a seed and planting the seed; applying mycobacteria to soil and planting a seed in the soil; applying mycobacteria to seedlings; applying mycobacteria to established plants; applying mycobacteria to the soil around established plants; and combinations thereof.

Additionally, phytobioremediation can be characterized by various methods that indicate the symbiotic relationship between the plant root and the mycobacteria. Examples of methods of characterizing and/or identifying phytobioremediation with mycobacteria can include the following: the presence of roots colonized by PAH-degrading mycobacteria improving the bioavailability of a model recalcitrant, such as pyrene; the mineralization of pyrene being enhanced by the interaction of the roots with the mycobacteria; detecting mycobacteria colonization of the root by detecting discrete interactions between the mycobacteria and root surface; testing the soil proximate to a root or the root to detect expression of the nid dioxygenase gene; culturing any microorganism in a soil sample in the presence of a selected contaminant, such as PAHs, PCPs, MTBEs, and other like contaminants; detecting a change in root activity; detecting a change in root phenoloxidase activity, where the root phenoloxidase may participate in PAH-remodeling; and combinations thereof. Thus, in order for phytobioremediation to be performed to decontaminate soils contaminated with PAHs, PCPs, MTBEs, and other like contaminants, contaminant-degrading mycobacteria, such as the mycobacteria strains JLS, KMS, MCS, and other mycobacteria having the nidB-nidA gene sequence, need to be identified, cultured, and placed in contaminated soils along with plant roots.

In its simplest state, termed rhizostimulation, components including sugars, peptides, glyco-complexes and phenolics in the plant root exudates provide nutrition for the growth of microbes that have bioremediant activity. Thus, the roots may maintain populations of the beneficial contaminant-degrading mycobacteria. Another benefit is that the plant roots can act as vectors for these microbes as they grow into the soil. Higher degrees of interaction may be involved where the plant itself can metabolize the pollutant or its microbially-transformed products. Plant laccases, cytochromes and peroxidases may be involved in these processes. Also, the microbes may aid in the initial steps in biodegradation or help to solubilize and/or metabolize the pollutant to make it more bioavailable to the plant

Microbial root colonization can involve several steps, such as: growth of cells in the rhizosphere and rhizoplane through utilization of the nutrients present in the root exudates, adhesion mediated by interactions between bacterial and root surface features, maturation of biofilm formation and possible ingress into internal tissues to become endophytic. Studies with root colonizing pseudomonads have shown utilization of root surface components. Attachment mechanisms that differ between legumes and other dicots have been demonstrated for Agrobacterium tumefaciens meaning that discrete surface structures are involved from both the plant and the microbe. Microbial extracellular polysaccharides are implicated in this and other colonization processes. Biofilm formation has been demonstrated with other Mycobacterium isolates of medical importance on artificial substrates. Complex signaling systems within the pseudomonads are demonstrated to be involved in biofilm formation and maturation.

Additionally, plant roots can secrete enzymes, such as peroxidases and laccases that have the potential to be involved in transformation of phenolic contaminants, such as those produced by microbial transformation of contaminants like PAHs, PCPs, MTBEs, and other similar contaminants. Also, it has been suggested that radicals are generated by such phenol-oxidizing activities from humic acids and that these then react with zenobiotics to cause their immobilization onto the humic materials. Indeed, amendments of soil with plant peroxidases may aid in pollutant remediation through immobilization. It is possible that oxidized breakdown products from a contaminant, such as PAH, mediated by the mycobacterium could be further metabolized by the plant's peroxidases, which requires hydrogen peroxide as a co-substrate, or laccases, which use molecular oxygen. Peroxidases are among enzymes that are modified, in activity or by isozyme composition, when plants are challenged by microbes or are stressed. It has been found that colonization of bean roots by Pseudomonas putida stimulated the production of a novel root surface peroxidase, and when wheat crowns were infected with Fusarium proliferatum there were changes in peroxidase isozymes.

Accordingly, the following can summarize some benefits of phytobioremediation: the presence of a mycobacterium-rhizosphere can be optimal for increasing the bioavailability of a contaminant, such as pyrene, to a plant; the mineralization of a contaminant, such as pyrene, can be enhanced by the rhizosphere-presence of mycobacterium; colonization of the root may involve discrete interactions between the mycobacterium and root surface; rhizosphere factors can influence the expression of the gene encoding the first enzyme involved in PAH degradation, dioxygenase, in the mycobacterium, wherein the expression of the gene can be an indication of successful phytobioremediation; and root phenoloxidases may change activity in roots that are colonized by mycobacterium that degrade contaminants.

II. Identifying Contaminant-Degrading Mycobacteria

Contaminant-degrading mycobacteria can be identified by placing samples, such as soil or root samples, on a medium having a selected contaminant and determining whether or not a mycobacteria culture can grow in the presence of the selected contaminant. As briefly stated, common culturing techniques can be extremely time consuming and can depend on factors unrelated to whether or not contaminant-degrading mycobacteria is present in the sample. Methods of identifying the contaminant-degrading mycobacteria that utilize the analysis of genetic material isolated from a sample can provide faster and more accurate detection methods. Thus detection methods using gene probes for the nidB-nidA gene sequence can be useful for detecting contaminant-degrading mycobacteria in soil samples.

A. Method of Preparing Soil Samples

In accordance with the present invention, samples can be prepared in order to determine whether or not they include PAH-degraders. Such samples can be prepared directly from soil that is in or around sites known to be contaminated with PAHs. The methods of sample preparation can be performed before subsequent genetic analysis, or prepared by an external source and then delivered to a facility for the genetic analysis as described below. While the soil can be collected from any location, it has been found that soil within or proximate to a site contaminated by a selected contaminant, such as PAHs, PCPs, MTBEs, and other like contaminants, can be a source of contaminant degraders such as mycobacteria that include nidB-nidA dioxygenase genes. Also, it is possible that additional strains of contaminant degraders can found in sites previously explored, such as the superfund site in Libby, Montana, or in sites that have not yet been explored. That is, a site that is known to be contaminated with a selected contaminant can be a source for samples to determine whether known contaminant degraders are present, or a source for identifying new contaminant degraders.

Additionally, the sample preparation method can include extracting genomic DNA from the soil. More particularly, this can include extracting genomic DNA from microorganisms, or more preferably, from mycobacteria. Extraction techniques for obtaining genomic DNA from soil are well known and described in more detail below and in the incorporated materials.

The sample preparation method can also include purifying the genomic DNA. That is, the purifying can remove impurities that can impede the ability to successfully produce a PCR product that conforms with the genome of mycobacteria present in the soil. For example, many types of proteinaceous, ionic, and hydrophobic substances can contaminate a PCR process. Purification techniques are well known and described in more detail below and in the incorporated materials.

Additionally, a method for preparing a sample from contaminated soil for genetic analysis can include sequential freezing and thawing of the sample so that the microorganisms are also frozen and thawed in repeated cycles. Freeze-thawing is a technique that has been shown to be effective during DNA extraction from microorganisms such as gram-positive bacteria. Further, the method can include bead beating the sample and microorganisms contained therein. Bead beating usually involves mixing the sample in the presence of glass beads, and is described in more detail below and in the incorporated materials.

The method can also include removing PCR inhibitors with binding resins. This usually includes passing the sample through a chromatographic column that is comprised of various resins that can selectively either pull the genomic DNA from the sample, or pull the contaminants or PCR inhibitors from the sample so as to remove the DNA from the contaminants. Binding resin chromatography of soil samples is described in more detail below and in the incorporated references.

While various methods of sample preparation have been described herein, it is contemplated that other methods of sample preparation can be employed in accordance with the present invention. In any event, the methods of testing samples for the presence of PAH-biodegrading mycobacteria are described in more detail below.

B. Methods of Identifying PAH-Biodegrading Mycobacteria

In accordance with the present invention, samples can be assayed in order to determine whether or not they include PAH-degraders. Methods of identifying contaminant-degrading mycobacterium that do not require culturing a sample on a medium with the contaminant can be beneficial. As such, contaminated soils can be assayed for contaminant-degrading microorganisms by extracting and purifying genomic DNA directly from the soil, and assaying the DNA for the presence of nidB-nidA dioxygenase genes indicative of the contaminant-degrading mycobacterium strains JLS, KMS, and MCS as well as others. Alternatively, the purified genomic DNA can be provided from another source without performing such an extraction (e.g., when another entity has already extracted the DNA from soil and merely wants to identify the presence of contaminant-degrading microbes).

In one embodiment, a method for determining whether a microorganism is capable of biodegrading a selected contaminant can be employed by assaying its genomic DNA. Such a method can be performed by providing a first set of DNA molecules consisting of fragments of genomic DNA of at least one mycobacteria species capable of biodegrading a selected contaminant, such as a PAH. The species can be the JLS, KMS, and MCS isolates previously identified, as well as others. As such, genomic DNA of these isolates, such as the nidB-nidA dioxygenase genes, can be employed to determine whether a soil sample includes mycobacterium with substantially similar genes. The presence of these types of genes is a strong indication that the soil contains microorganisms that biodegrade PAHs.

Additionally, the method can also include contacting, under hybridizing conditions, the first set of DNA molecules with a second set of DNA molecules consisting of genomic DNA of an unknown microorganism. More particularly, it is not known whether or not the unknown microorganism biodegrades a selected contaminant. The hybridizing conditions can range from low, medium, and high stringency so that the ability of the probe to hybridize with the unknown genomic DNA can be modulated. This is because the stringency conditions can determine the ability of the probe (e.g., first set of DNA molecules) to properly hybridize with the genomic DNA of the unknown microorganism, which can range from partial hybridization through full hybridization where each nucleotide in the probe associates with the complement nucleotide in the genomic DNA.

The method can also include detecting hybridization between the first set of DNA molecules and the second set of DNA molecules. The detecting can include producing a PCR product and comparing the nucleotide sequence of the PCR product with nidB-nidA dioxygenase genes by electrophoresis, sequencing, gene-chip, or other well-known means. Also, a gene-chip can be used without performing PCR. Hybridization between the first and second sets of DNA is an indication that the microorganism is capable of biodegrading the selected contaminant. This is because the genomic DNA of the unknown microorganism is likely to code for nidB-nidA dioxygenase enzymes when hybridization occurs. More particularly, when the first set of DNA molecules can hybridize with the second set of DNA molecules, it is likely that nidB-nidA dioxygenase gene sequence is conserved between the known contaminant-degrading mycobacterium and the unknown microorganism. Thus, such hybridization indicates the unknown microorganism is also a capable of degrading the selected contaminant, and can be a contaminant-biodegrading mycobacterium.

Additionally, another method for identifying contaminant-degrading mycobacteria can be performed by providing at least one primer or primer set capable of hybridizing with a nidB-nidA dioxygenase genomic DNA nucleotide sequence of at least one known PAH-degrading mycobacteria. As such, the preparations of primers and nucleotide sequences that can hybridize with a nidB-nidA dioxygenase genomic DNA nucleotide sequence are described in more detail below. Additionally, the method can include contacting at least one primer or primer set with a sample. More particularly, the contacting can be performed with a sample of genomic DNA isolated from soil as described herein, where the genomic DNA has been purified so that it can be used in PCR. This is because substances in an unpurified sample, such as proteinaceous or other substances, can contaminate the PCR and result in inaccurate PCR products. The method can also include producing a PCR product. A PCR product can be produced by any of the well-known PCR methods as well as those subsequently developed. Briefly, PCR products can be obtained by annealing the primer to genomic DNA complement thereto and introducing a polymerase capable of adding nucleotides to the primer so as to become a complement of the genomic DNA to which it has annealed. Further, the method can include determining whether the PCR product indicates the presence of genomic DNA of a microorganism having a nidB-nidA dioxygenase nucleotide. Such a determination can be made when the PCR product is similar to known nidB and/or nidA dioxygenase gene sequences. That is, when the PCR product is comparable to known nidB and/or nidA nucleotide sequences, it is indicative that the sample, such as a soil sample or sample prepared therefrom, includes a mycobacterium capable of degrading PAHs.

In one embodiment, any of the foregoing methods can include performing a PCR to amplify the amount of a second set of DNA molecules (e.g., DNA isolated from soil) as at least a portion of the method for determining whether a microorganism is capable of biodegrading PAHs or whether a sample includes such microorganisms. As such, a first portion of a first set of DNA molecules (e.g., PAH-degrading mycobacteria genomic DNA molecules) includes a plurality of primers. That is, primers, primer sets, and/or primer pair can be prepared from contaminant-degrading mycobacteria genomic DNA molecules. Each of the primers can be comprised of a primer nucleotide sequence having from about 8 to about 30 nucleic acids, more preferably from about 19 to about 25, and most preferably about 21 nucleic acids.

In one embodiment, the determination of whether or not known genomic DNA indicates the presence of contaminant-biodegrading mycobacteria includes comparing the size of the. PCR product with a DNA ladder by performing electrophoresis. Also, electrophoresis can compare the PCR product with known nidB DNA, nidA DNA, and/or combinations thereof. Furthermore, electrophoresis can use known nidB DNA and/or nidA DNA from at least one of JLS, KMS, or mycobacterium MCS.

In one embodiment, the determination of whether or not known genomic DNA indicates the presence of a contaminant-degrading mycobacterium includes sequencing the PCR product to determine the nucleotide sequence thereof. Sequencing is an established and well-known technique that provides the sequence of the nucleic acids. Additional information on sequencing protocols can be found in the incorporated materials and elsewhere.

In any event, after the sequence of the PCR product is obtained, the sequence can be compared with a known contaminant-degrading mycobacterium nidA and/or nidB nucleotide sequence. Also, the sequence can be compared with a nidA and/or nidB nucleotide sequence from a known contaminant-degrading mycobacterium selected from the group consisting of JLS, KMS, MCS, Mycobacterium vanbaalenii, Mycobacterium frederiksbergense strain FAn9T, Mycobacterium flavescens strain PYR-GCK. Also, it is contemplated that the PCR product sequence can be compared to future-discovered contaminant-degrading mycobacterium genomic DNA sequences.

In one embodiment, comparing the PCR product nucleotide sequence with a known PAH-degrading mycobacterium nidA and/or nidB nucleotide sequence can result in a substantially homologous or conserved nucleotide sequence between the unknown mycobacteria and, the known PAH-degrader. As such, a nucleotide identity match greater than 95% indicates the sample, such as a soil sample or microorganism sample, contains a polycyclic aromatic hydrocarbon-degrading mycobacterium. More particularly, the nucleotide identity match is greater than 97%, and most preferably, 99% or greater.

Additional information regarding identifying contaminant-degrading mycobacteria can be found in the incorporated references.

III. Mycobacteria Compositions

A mycobacterium, or other microorganism, identified as being capable of degrading a selected contaminant can be formulated into a composition for use in bioremediation of soil contaminated with the selected contaminant. While the following generally discloses and describes compositions having mycobacteria, it should be recognized that any microorganism capable of degrading a selected contaminant can also be used. Moreover, while the following generally discloses mycobacteria, such as JLS, KMS, and MCS, capable of degrading PAHs, such mycobacteria can be used to bioremediate soil contaminated with PCPs, MTBEs, and other like contaminants.

A. Mycobacteria Medium

Generally, mycobacteria compositions can include mycobacteria in a solution. Preferably, the solution includes water, and can also include media to support the growth and propagation of mycobacteria. Such media can be formulated to include ingredients that are favorable to mycobacteria, and preferably favorable to the JLS, KMS, and MCS strains.

Studies have been performed to determine favorable substances that can be included in a medium designed to grow and propagate mycobacteria. Based on the carbon preferences of the mycobacteria JLS, KMS, and MCS strains, selected carbon sources can be advantageously combined with a mycobacteria medium. The carbon sources can include D-mannitol, D-psicose, propionic acid, D-sorbitol, sucrose, alpha-cyclodextrin, and sedoheptulosan. More preferably, the carbon sources can include polyoxyethylene sorbitan mono-palmitate (Tween 40), polyoxyethylene sorbitan monooleate (Tween 80), D-fructose, D-mannose, D-trehalose, and pyruvic acid methyl ester. Additionally, beneficial substances can include complex mixtures containing polysaccharides and other nutrients (e.g., molasses, whey effluent, and the like).

However, certain carbon sources have been identified as unfavorable for a mycobacteria medium, or can be included in minimal quantities so as to prevent injury to the mycobacteria. As such, less favorable carbon sources that can be excluded from a mycobacteria medium can include dextrin, L-arabinose, D-arabitol, D-cellobiose, D-gluconic acid, alpha-D-glucose, alpha-methyl-D-glucose, xylitol, erythritol, acetic acid, alpha-hydroxybutric acid, beta-hydroxybutric acid, lactamide, succinic acid, N-acetyl-L-glutamic acid, and glycerol.

In one embodiment, the medium designed for growing and propagating the contaminant-degrading mycobacteria can include roots, root extracts, root exudates, root pulp, and liquefied root. In part, this is because the sugars, peptides, glyco-complexes, plant laccases, cytochromes, enzymes, peroxidases, and phenolics in the plant root and plant root exudates can provide nutrition for the growth of the mycobacteria and any enzymes from the plant may aid in the degradation process. It has been determined that the roots capable of providing support for mycobacteria in phytobioremediation can also be used to supplement a medium for growing the mycobacteria. Thus, the roots may maintain populations of the beneficial mycobacteria which can subsequently be processed into a form to be used in a medium. Another benefit is that the plant roots can act as vectors for these microbes as they grow into the soil. Higher degrees of interaction may be involved where the plant itself can metabolize the pollutant or its microbially-transformed products.

While the medium for growing and propagating mycobacteria can be prepared from the roots of most plants, certain plants that can support mycobacteria in soil can be preferred. Accordingly, preferred roots to be utilized in preparing a mycobacteria medium can include barley, wheatgrass, Lolium species, legumes, alfalfa, rice, grasses, forbs, trees, mulberry tree, clover, brassicas, curcubits, and rye.

Additionally, the medium for growing and propagating mycobacteria can be prepared with any commercial medium for use with bacteria or mycobacteria. Preferably, the medium includes Middlebrooks. Additionally, the medium can include various substrates and additives that are well known in the art of bacteria or mycobacteria medium preparation. Also, it is preferable for the medium to be sterile before inoculation with mycobacteria.

After a suitable medium is prepared and sterilized, the contaminant-degrading mycobacteria can be grown and propagated. The medium can be used in an amount suitable for growing the mycobacteria.

B. Mycobacteria-Coated Seeds

In one embodiment, a seed can be coated with mycobacteria. As such, the seed can then be planted in contaminated soil so that the mycobacteria can grow and colonize on the root of the plant that grows from the seed. A seed coated with mycobacteria can be prepared in different configurations.

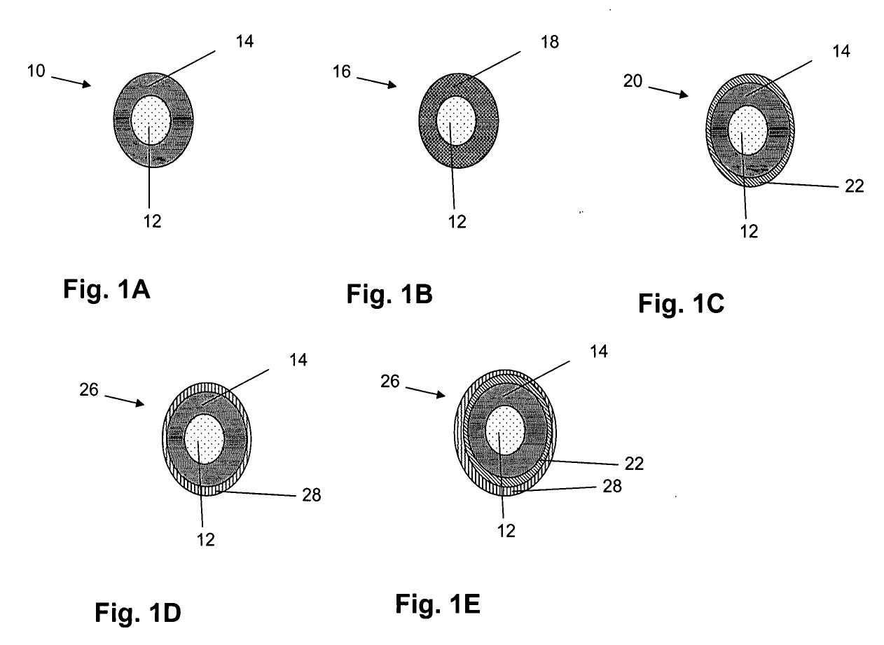

FIG. 1A illustrates one embodiment of a mycobacteria-coated seed 10. As such, the mycobacteria-coated seed 10 can include the seed 12 having the mycobacteria 14 adhered thereto. For example, the mycobacteria can be adhered to the seed by dipping the seed in a mycobacteria solution. The seed can then be planted while the seed is wet with the mycobacteria solution, or the seed can be dried and planted at a later time.

FIG. 1B illustrates another embodiment of a mycobacteria-coated seed 16. In some instances it can be beneficial for the seed 12 to have a matrix coating 18 comprising the mycobacteria. As such, the mycobacteria solution can include an ingredient that facilitates adherence to the seed. For example, such ingredients can include natural gums, gum karaya, xanthum gum, gum arabic, gum tragacanth, polysaccharides, starches, celluloses, amyloses, inulins, chitins, chitosans, amylopectins, glycogens, pectins, hemicelluloses, glucomannans, galactoglucomannans, xyloglucans, methylglucuronoxylans, arabinoxylans, methylglucuronoarabinoxylans, glycosaminoglycans, chondroitins, hyaluronic acids, alginic acids and the like. Preferably, the matrix is biodegradable.

FIG. 1C illustrates yet another embodiment of a mycobacteria-coated seed 20. In some instances, it can be beneficial for the seed 12 to have the mycobacteria 14 adhered thereto, and coated with a biodegradable polymer coating 22, which can allow for extended storage. The biodegradable polymer coating can include at least one of poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like.

FIG. 1D illustrates yet another embodiment of a mycobacteria-coated seed 26. In some instances, it can be beneficial for the seed 12 to have the mycobacteria 14 adhered thereto, and coated or partially coated with a water-resistant polymer coating 28, which can allow for extended storage. For example, the water-resistant polymer can include ethylene-vinyl alcohol copolymer (“EVOH”), ethylene-vinyl acetate copolymer (“EVA”), propylene-vinyl alcohol copolymer (“PVOH”), propylene-vinyl acetate copolymer, polyvinyl alcohol (“PVA”), partially hydrolyzed ethylene-vinyl acetate copolymer, propylene-vinyl alcohol, and the like. Typically, the water-resistant polymer coating 28 is extremely thin, and serves to protect the seed 12 and mycobacteria 14 during transportation. Even through the coating is water-resistant, exposure to soil can cause the coating to rupture, become cracked or fissured so that a seedling and the mycobacteria are able to grow.

FIG. 1E illustrates yet another embodiment of a mycobacteria-coated seed 30. In some instances it can be beneficial for the seed 12 to have the mycobacteria 14 adhered thereto, and coated with a biodegradable polymer coating 22, and then further coated or partially coated with a water-resistant polymer coating 28.

C. Mycobacteria Pellets

In one embodiment, a composition including at least one mycobacterium with or without a suitable growth medium can be prepared into a solid form. For example, a solution having mycobacteria and suitable growth medium can be combined with thickeners or matrix ingredients, and then solidified into a biodegradable form. The solid preparation can be prepared by drying or by including a matrix ingredient or biodegradable polymer in an amount to form a solid. A solid composition including a mycobacterium and suitable growth medium can then be cut, milled, or pelletized into solids of an appropriate size. Preferably, the solid is pelletized into pellets that can be easily delivered to soil.

D. Mycobacteria Fertilizer

In one embodiment, a composition including at least one mycobacterium with or without a suitable growth medium can be included in a fertilizer. As such, the composition can be included in any standard fertilizer by being added as a liquid or a solid. Preferably, a pellet comprising the mycobacteria with or without a suitable growth medium is prepared and added to an existing solid or liquid fertilizer.

IV. Methods of Phytobioremediation

Generally, the present invention can include methods of phytobioremediation that utilize contaminant-degrading mycobacteria colonized on a plant root. Preferably, the mycobacteria is a JLS, KMS, and MCS strain. More preferably, the mycobacteria includes nidB-nidA dioxygenase gene sequences. Thus, a plant root-mycobacteria system can be used to degrade contaminants, such as PAHs, PCPs, MTBEs, and like contaminants.

The plant root can be from any plant capable of growing in the contaminated soil. For example, the plant root can be from barley, wheatgrass, Lolium species, legumes, alfalfa, rice, grasses, forbs, trees, mulberry tree;.clover, corn, rye, curcubits, brassicas, or other plant found growing in a location contaminated with a selected contaminant. It has been demonstrated that mycobacteria can colonize barley roots after seed inoculation and planting in a sterile medium or soils containing natural microflora. Barley has been selected and used as the plant host because it can survive in PAH-contaminated soil, and high salt contaminated soil.

In one embodiment, phytobioremediation can be performed by planting a mycobacteria-coated seed into contaminated soil. Accordingly, any process for planting seeds can be used to plant the mycobacteria-coated seed. This can include seeds having a dry or wet mycobacterial coating. Preferably, the seeds are planted with a dry coating. Alternatively, the seed can be dipped into a solution containing the mycobacteria, and then planted while wet.

In another embodiment, a seed can be planted into contaminated soil and then the soil can be inoculated with a composition containing the mycobacteria. This can include the composition being in a liquid or solid form. For example, a liquid can be sprayed on the soil, and pellets can be sprinkled on the soil. Also, fertilizer having the mycobacteria can be used to fertilize the soil.

In another embodiment, a seedling can be grown in uncontaminated soil and then transplanted into contaminated soil. The seedling can be inoculated with a composition containing the mycobacteria before or after being transplanted. This can include planting a mycobacteria-coated seed into uncontaminated soil, growing the seedling, and then transplanting the seedling into the contaminated soil.

In another embodiment, a seedling can be grown in contaminated soil and then inoculated with a composition containing the mycobacteria. This can include planting a seed into contaminated soil, growing the seedling, and then inoculating soil with a composition containing the mycobacteria. Alternatively, the seedling can be a pre-existing plant in the contaminated soil.

In another embodiment, a plant can be grown in uncontaminated soil and then transplanted into contaminated soil. The soil around the plant can be inoculated with a composition containing the mycobacteria before or after being transplanted. This can include planting a mycobacteria-coated seed into uncontaminated soil, growing the plant, and then transplanting the plant into the contaminated soil.

In another embodiment, a plant can be grown in contaminated soil and then inoculated with a composition containing the mycobacteria. This can include planting a seed into contaminated soil, growing the seedling, and then inoculating soil with a composition containing the mycobacteria. Also, plants preexisting in contaminated soil can be inoculated with a composition containing the mycobacteria.

Additionally, throughout the phytobioremediation, assays can be conducted to determine whether or not the contaminant-degrading mycobacteria have colonized on a plant root. In instances where additional mycobacteria may be desired, the soil can be re-inoculated with mycobacteria. In instances where the mycobacteria colonization is less than desired, the soil can be re-inoculated with either mycobacteria or a suitable medium for growing the mycobacteria as described herein.

### EXAMPLES OF EMBODIMENTS OF THE INVENTION

The following examples illustrate embodiments of the present invention that can be employed in order to facilitate soil decontamination by phytobioremediation. Additionally, experiments for identifying the presence of a contaminant-degrading mycobacteria are described in the incorporated references.

### Example 1

An example of soil identified to include PAH-degraders includes the PAH-contaminated soil from the land treatment unit (“LTU”) at the Champion International Superfund Site in Libby, Montana. The soil was characterized as a loam (48% sand, 39% silt and 13% clay). The soil had a pH of 6.6, an EC of 4.5 mhos/ cm, and 1.88% organic carbon. The soil was passed through a 1.7 mm sieve and homogenized by hand and was stored in the dark at 4° C. until it was used. The soil had a moisture content of 10.2%. As such, it contemplated that various other types of soil can also include PAH-degraders.

The LTU soil was processed in order to assess the presence of PAH-degraders. Briefly, colonies capable of degrading pyrene were obtained from the LTU soil by suspending samples (0.1 g/ml) in sterile distilled water followed by serial dilution and spreading onto a basal salts medium (“BAM”) containing mineral nutrients but no carbon source. The basal salts medium contained (in 1 liter): 2.38 g (NH₄)SO₄, 0.28 mg FeSO₄*7H₂O, 10.69 mg CaCl₂*7H₂O, 0.25 g MgSO₄*7H₂O, 0.50 g NaCl, 1.42 g Na₂HPO₄, 1.36g KH₂PO₄, pH 6.5. Agar was added at 1.5%. The plates were airbrushed with a solution of pyrene in hexane/acetone (1:1) until an opaque layer had formed on the surface. The inoculated plates were placed in an incubator at 30° C. and bacteria were allowed to form visible colonies. The colonies producing a clear zone in the opaque layer were transferred to tryptic soy agar plates for single colony isolation. Four types of bacteria isolates were initially isolated using this technique, three of which were used for subsequent studies. For storage, cells of these three bacteria, the JLS, KMS, and MCS strains, were grown in Luria broth (“LB”) (Difco; Becton, Dickinson; Sparks; and MD) cultures and were suspended in 15% glycerol before being stored at −80° C. Liquid media cultures were generated from freezer stocks in BSM+(9:1 mixture v/v of BSM and LB) by shaking at 220 rpm at 25° C. Five-day-old cultures were used for analysis and various inoculations. Additionally, utilization of phenathrene and benzo(a) pyrene by isolates JLS, KMS, and MCS was determined using BSM plates possessing an overlay of these materials as described above.

The results indicated that the three bacteria from the PAH-contaminated LTU, soil formed PAH-degrading bacterial colonies surrounded by zones of clearing of pyrene layered onto BSM-agar plates. Each isolate grew rapidly in broth culture on LB media. All three isolates were gram positive, although they had different cell morphologies. Isolate JLS was a coccus, KMS a short rod, and MCS a long rod.

### Example 2

Experiments were conducted to determine substances that can be used in media to support growth and propagation of contaminant-degrading mycobacteria. As such, strains of M. KMS, JLS and MCS from the Libby sites and strains M. flavescens (“flav”) and M. vanbalenni (“PYR-1”) along with a standard M. smeginatis (“smeg”) were grown on media composed of various substances. Table 1 shows the substrates used by M. KMS, JLS and MCS from the Libby sites.

TABLE 1

| | KMS | MCS | JLS |
|---|---|---|---|
| Common Substrates | | | |
| Tween 40 | x | x | x |
| Tween 80 | x | x | x |
| D-Fructose | x | x | x |
| D-Mannose | x | x | x |
| D-Trehalose | x | x | x |
| Pyruvic Acid Methyl Ester | x | x | x |
| Differential Substrates | | | |
| D-Mannitol | x | | |
| D-Psicose | x | | |
| Propionic acid | | x | |
| D-Sorbitol | x | | |
| Sucrose | x | | |
| α-Cyclodextrin | | | x |
| Sedoheptulosan | x | | |

Table 1 shows that the tweens, mannose, fructose, trehalose, and pyruvic acid methyl ester were commonly used by the M. KMS, JLS and MCS strains and could be formulated to boost mycobacterium inocula over other bacteria. As such, these compounds can be used as carbon sources for growing and propagating the contaminant-degrading mycobacteria.

Table 2 shows the substrates used by M. KMS, M. flavescens (“flav”) and M. vanbalenni (“PYR-1”) along with a standard M. smegmatis (“smeg”) were grown on media composed of various substances.

TABLE 2

| | KMS | Pyr-1 | flav | smeg |
|---|---|---|---|---|
| Common Substrates | | | | |
| Tween 40 | x | x | x | x |
| Tween 80 | x | x | x | x |
| D-Fructose | x | x | x | x |
| D-Mannose | x | x | x | x |
| Sedoheptulosan | x | x | x | x |
| D-Sorbitol | x | x | x | x |
| Novel Substrates | | | | |
| Dextrin | | | | x |
| L-Arabinose | | | | x |
| D-Arabitol | | | | x |
| D-Cellobiose | | | | x |
| D-Gluconic Acid | | | | x |
| α-D glucose | | | | x |
| 3-Methyl-D-Glucose | | | | x |
| α-Methyl-D-Glucoside | | | | x |
| Xylitol | | | | x |
| Acetic Acid | | | | x |
| α-Hydroxybutyric Acid | | | x | |
| β-Hydroxybutyric Acid | | | | x |
| Lactamide | | | | x |
| Succinic Acid | | | | |
| N-Acetyl-L-Glutamic Acid | | | x | |
| Glycerol | | | | x |

Based on Tables 1 and 2, the identification of carbon preferences by M. KMS, M. flavescens (“flav”) and M. vanbalenni (“PYR-1”) along with a standard M. smegmatis (“smeg”) can be used to select carbon sources that can be advantageously combined with a mycobacteria medium for M. KMS, JLS and MCS strains. The carbon sources can include D-mannitol, D-psicose, propionic acid, D-sorbitol, sucrose, alpha-cyclodextrin, and sedoheptulosan. More preferably, the carbon sources can include Tween 40, Tween 80, D-fructose, D-mannose, D-trehalose, and pyruvic acid methyl ester.

However, certain carbon sources have been identified as unfavorable for use in a mycobacteria medium for M. KMS, JLS and MCS strains, or may be included in minimal quantities so as to prevent injury to the mycobacteria. As such, less favorable carbon sources that can be excluded from a mycobacteria medium can include dextrin, L-arabinose, D-arabitol, D-cellobiose, D-gluconic acid, alpha-D-glucose, alpha-methyl-D-glucose, xylitol, erytlrritol, acetic acid, alpha-hydroxybutric acid, beta-hydroxybutric acid, lactamide, succinic acid, N-acetyl-L-glutamic acid, and glycerol.

### Example 3

Studies were conducted to determine whether or not mycobacteria are capable of forming a biofilm in the presence of a root wash. Briefly, isolated M. KMS, JLS and MCS strains, M. flavescens (“flav”), and M. vanbalenni (“PYR- 1”) were grown in the presence of Middlebrooks medium or a barley root wash. Liquid medium (e.g., root wash and commercial Middlebrooks) were inoculated and grown with shaking for 10 days. Initial inocula were 10⁵-10⁶ cfu/mL. The mycobacteria where then analyzed to determine the extent of biofilm formation.

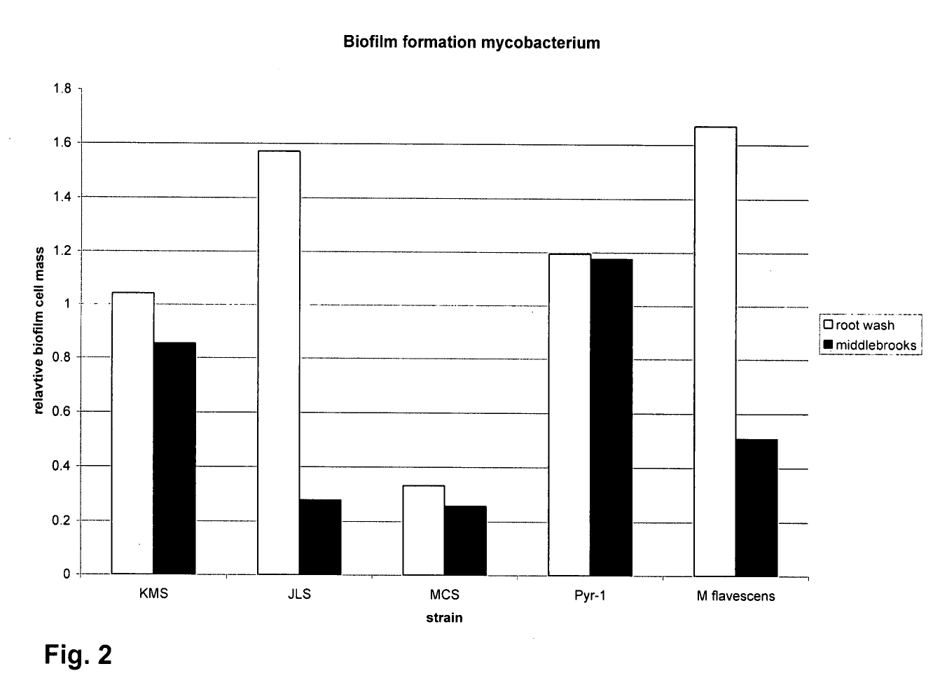

FIG. 2 shows that different mycobacterium strains have different potential for biofilm formation in the presence of root wash compared to Middlebrooks medium. Root wash permitted better biofilm formation compared to a complex commercial medium called Middlebrooks. More particularly, the isolated M. KMS, JLS and MCS strains showed enhanced biofilm formation in root wash compared to Middlebrooks medium. Thus, the substances within a root can be useful for growth and propagation of mycobacteria, especially for the isolated M. KMS, JLS and MCS strains.

### Example 4

Studies were conducted to determine whether or not mycobacteria are capable of planktonic growth in the presence of a root wash. Briefly, isolated M. KMS, JLS and MCS strains, M. flavescens (“flav”), and M. vanbalenni (“PYR-1”) were grown in the presence of Middlebrooks medium or a barley root wash. Liquid medium (e.g., root wash and commercial Middlebrooks) were inoculated and grown with shaking for 10 days. Initial inocula were 10⁵-10⁶ cfu/ml. The mycobacteria where then analyzed to determine the extent of planktonic growth.

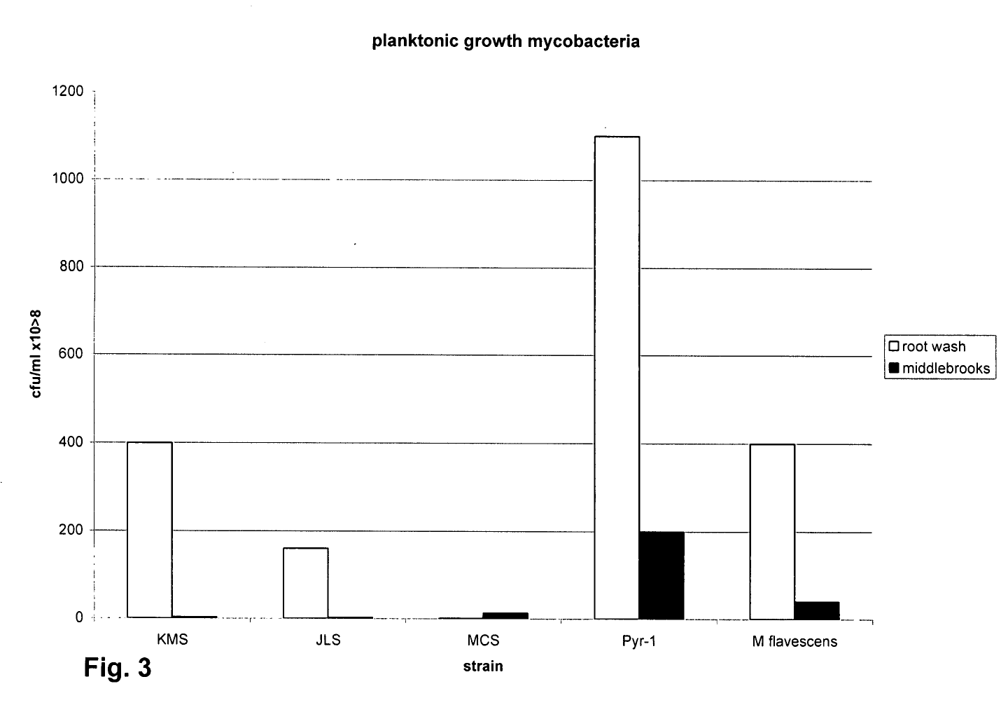

FIG. 3 shows that different mycobacterium strains have different potential for planktonic growth in the presence of root wash compared to Middlebrooks medium. The isolated M. KMS and JLS strains showed enhanced planktonic growth in root wash compared to Middlebrooks medium. On the other hand, MCS did not show enhanced planktonic growth. Thus, the substances within a root can be useful for growth and propagation of mycobacteria, especially for the isolated M. KMS, JLS and MCS strains. Also, there were differences in the final cell densities between the strains with PYR-1 being greater than KMS and M. flavescens.

### Example 5

It has been demonstrated that the mycobacteria M. KMS, JLS and MCS strains can colonize barley roots after seed inoculation and planting into sterile medium or soils containing natural microflora. Strong colonization is apparent in five-day-old inoculated seedlings.

### Example 6

Studies were conducted to determine whether or not mycobacteria can present strong colonization of plant roots in soils with native microbes present. Briefly, barley seeds were inoculated with the Libby M. KMS, by immersion into a suspension of 10⁹ colony forming units/mL, or were planted without inoculation. Seeds were planted at a depth of 1 inch into either background uncontaminated soil or PAH-contaminated soil from the Libby, MT site. After 14 days, roots were removed gently, vortexed in 5 mL sterile water for 1 minute and dilution plated onto Kings medium B agar plates with and without rifampicin and tetracycline to determine KMS and total bacterial colonies.

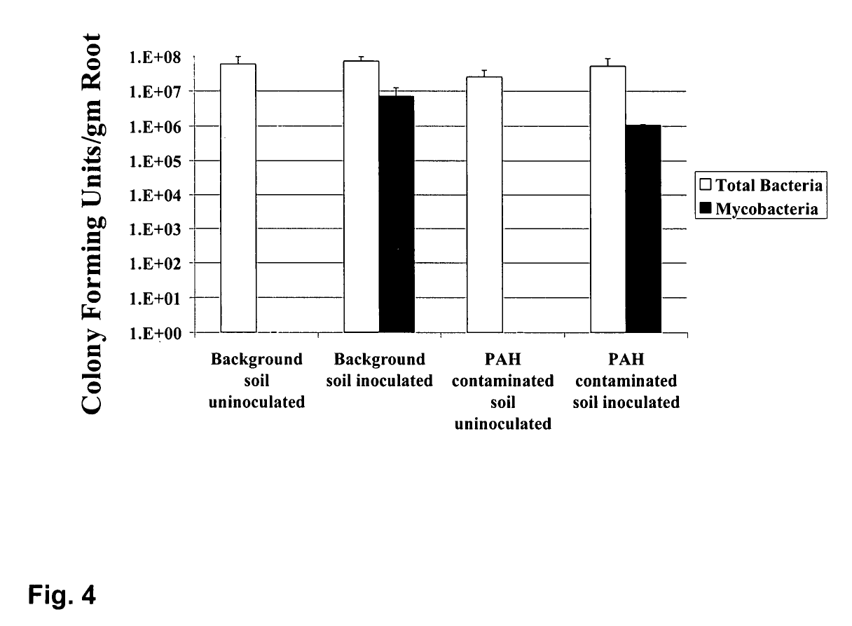

FIG. 4 shows the Libby mycobacterium isolate KMS colonized barley roots from a seed-borne inoculum. Similar findings are obtained with the other two Libby isolates. The mycobacterium was recovered from the roots at high levels in relation to total cell recovery after growth for ten days in a soil containing a normal microbial load (10⁷⁻⁸ cfu/g).

### Example 7

Seeds can be prepared prior to being coated with a mycobacteria composition. Briefly, seeds were processed to remove endogenous surface microbes and microbial endophytes. Seeds were immersed in 30% hydrogen peroxide for 5 minutes and washed with sterile water for three minutes, followed by three subsequent one-minute washes with sterile water to remove any remaining hydrogen peroxide. The seeds were heat-treated by suspension in sterile water at 50 ° C. for 30 minutes. After the heat treatment step, the seeds were surface-sterilized again following the hydrogen peroxide method previously described.

Treated seeds were plated on LB agar plates and incubated at 22° C. for up to 48 h to permit germination and detection of fungal or bacterial contamination. Seeds that showed signs of microbial contamination were discarded. Clean seeds were inoculated by submersing them in a suspension of mycobacterium cells for 30 seconds.

### Example 8

Seedlings can be grown in sterile environments. Briefly, the inoculated seeds of Example 7 were tested planted in sterile vermiculite for seedling growth. This growth matrix was prepared by adding 125 mL sterile water to approximately 325 mL vermiculite in Magenta boxes, and sterilizing at 121 ° C. for 40 minutes. After storing at room temperature for 24 h to allow fungal and bacterial spore germination, the boxes were sterilized again at 121° C. for 40 minutes. Three seeds were planted per container, and the plants were grown gnotobiotically at 26° C.

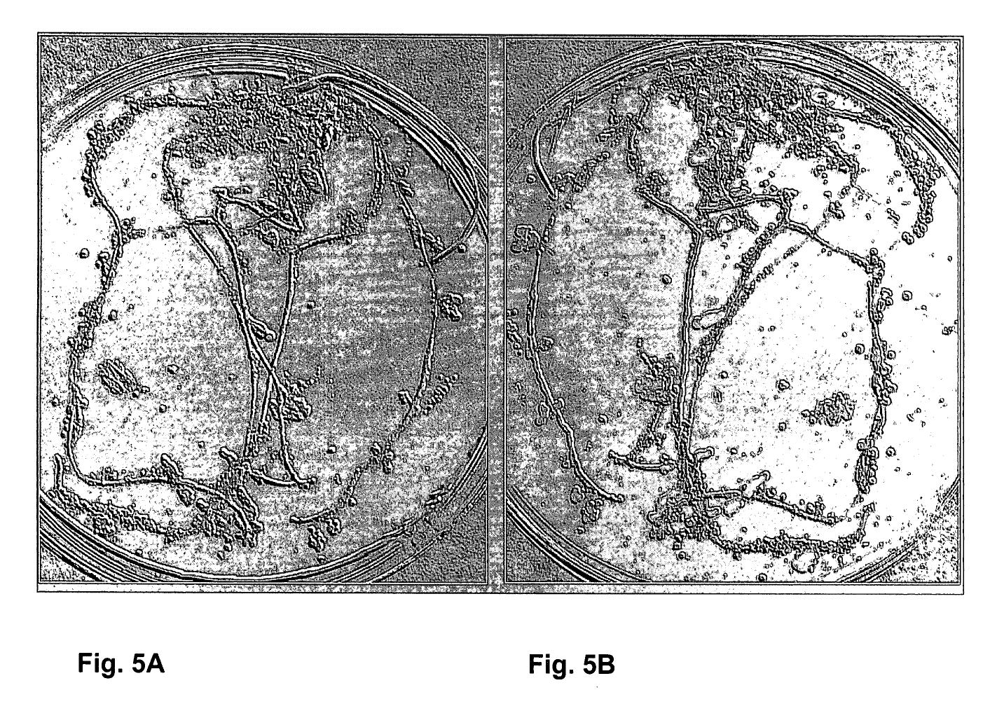

Strong colonization is apparent in 7 day old inoculated seedlings. This was determined by harvesting roots at 7 days, and blotted the root onto LB plate medium. The roots were incubated for 15 days before photographs were taken of the colonies. FIG. 5A is a top view of the colonies forming around the root, and FIG. 5B is a bottom view. PCR was used to confirm the identity of bacterial colonies as mycobacteria.

### Example 9

Barley seeds were prepared to include mycobacterium adhered to the outer surface of the seed. Briefly, cells were grown on amended Middlebrook 7H9 liquid medium. The cells were harvested during log-phase growth after five days, washed twice in sterile water, and suspended in sterile water. To determine the number of mycobacterium cells adhering to each seed, barley seeds inoculated as previously described were submersed in 1 mL sterile water and vortexed for 30 seconds. Serial dilutions of the water fractions were then performed and cfu/mL of cells were determined. The final cell density of the inoculum was approximately 10⁸ cfu/mL.

Example 10

Different sections of roots grown from barley seeds coated with mycobacteria were assayed to determine whether different sections of roots were better at sustaining mycobacteria growth. Briefly, roots that were not used in direct planting were harvested and dissected into 2 cm sections and vortexed in 1 mL sterile water for 30 seconds. Serial dilutions were made from the water onto LB plate medium and the number of mycobacterium colonies was determined for the different root sections. PCR was used to confirm the identity of the colonies. Serial dilutions from root sections of uninoculated sterile control seedlings were performed, and no microbial contamination was observed.

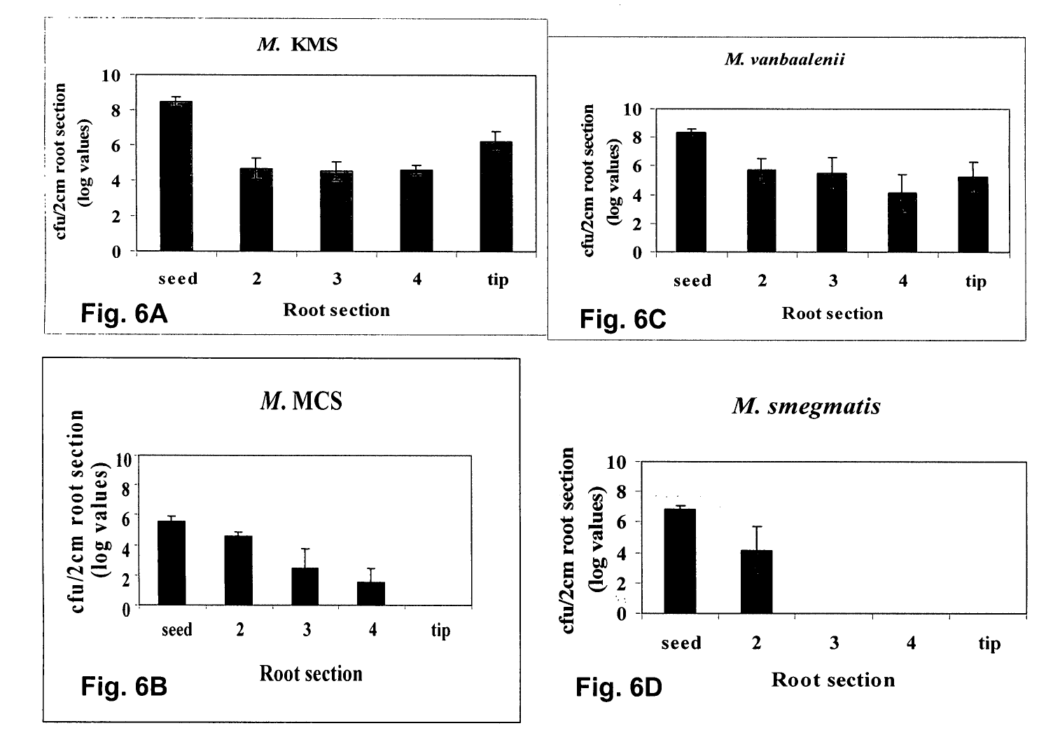

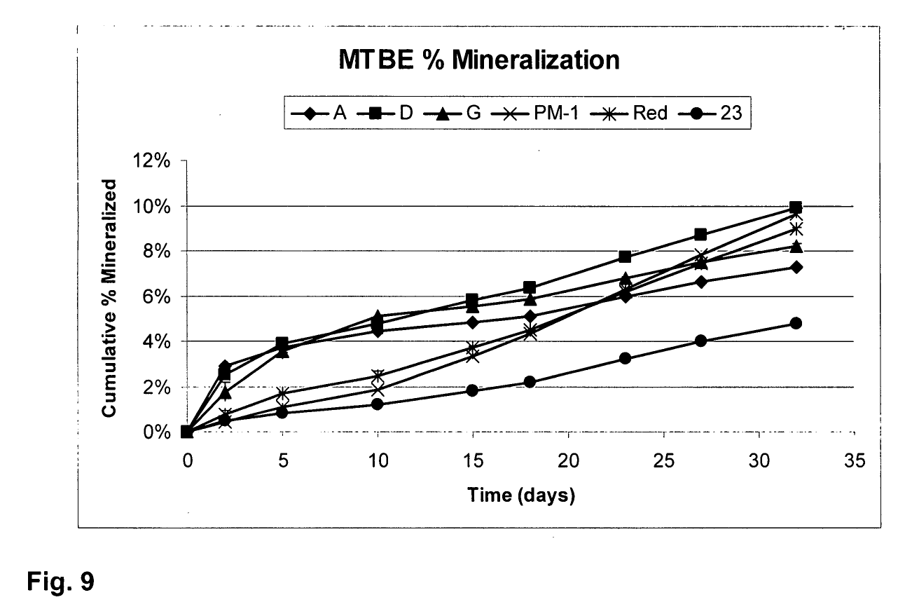

FIGS. 6A-61D show the colonization of mycobacteria on different root sections. Both KMS and M. vanbalenni show colonization of the root tip, which is a classic indication of a strong colonizing mycobacteria. As such, FIGS. 6A-6D show that the root may serve as a type of bioinjector, and can be used to transport bacteria to different soil levels and pockets of contamination.

### Example 11

Studies were conducted to compare phytobioremediation against phytoremediation and bioremediation. As such, four conditions were tested as follows: sterile, uninoculated radiolabeled pyrene-amended sand (control); uninoculated barley only; M. KMS only; and barley inoculated with M. KMS. Each of the conditions was grown in a closed environment. Briefly, air was pumped through the system for 4 hours every 24 hour period, and 1 mL samples of a CO₂ trap solution were taken every two days and radioactivity counts were read using a scintillation counter. The experiment period was 10 days. After the experiment was terminated, ¹⁴C levels in the barley roots and leaves were determined by combustion and ¹⁴CO₂ collection. The ¹⁴C amounts in the sand were also determined by combustion.

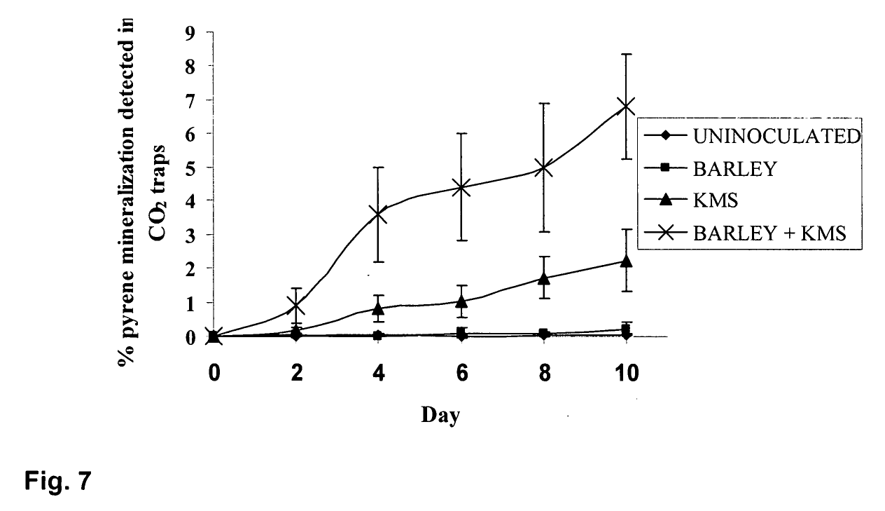

FIG. 7 shows that the phytobioremediation using barley and M KMS was superior to phytoremediation with barley and bioremediation with M. KMS (the data shown in FIG. 7 are the mean of three independent experiments ± standard deviations). Also, bioremediation was superior to phytoremediation.

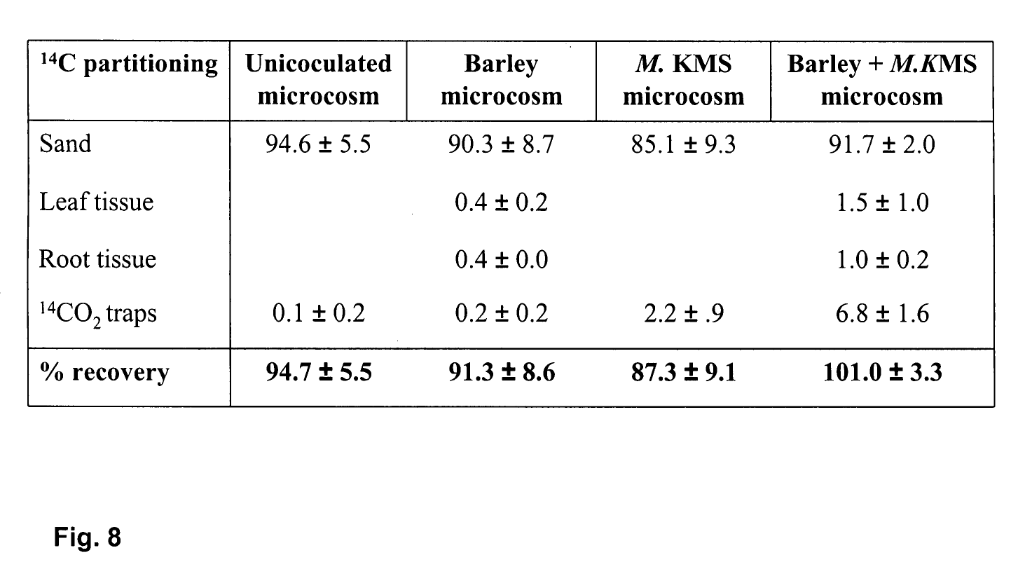

FIG. 8 is a mass balance of ¹⁴C. As such, the table shows that the ¹⁴C was preferentially relocated from the soil to the ¹⁴CO₂ collection traps compared to roots and leaves.

### Example 12

The mycobacteria M. KMS, JLS, and MCS strains were tested for the ability to mineralize MTBE and tertbutyl acetate (“TBA”), and compared against the mineralization ability of mycobacterium PM-1, and two bacteria cultured from a Ronan site. Microcosms were incubated statically in the dark at 32° C. for optimum temperature. Controls included one pure culture of each microbe was not spiked with MTBE or TBA. Concentrations of MTBE and TBA were set at 5 mg/L.

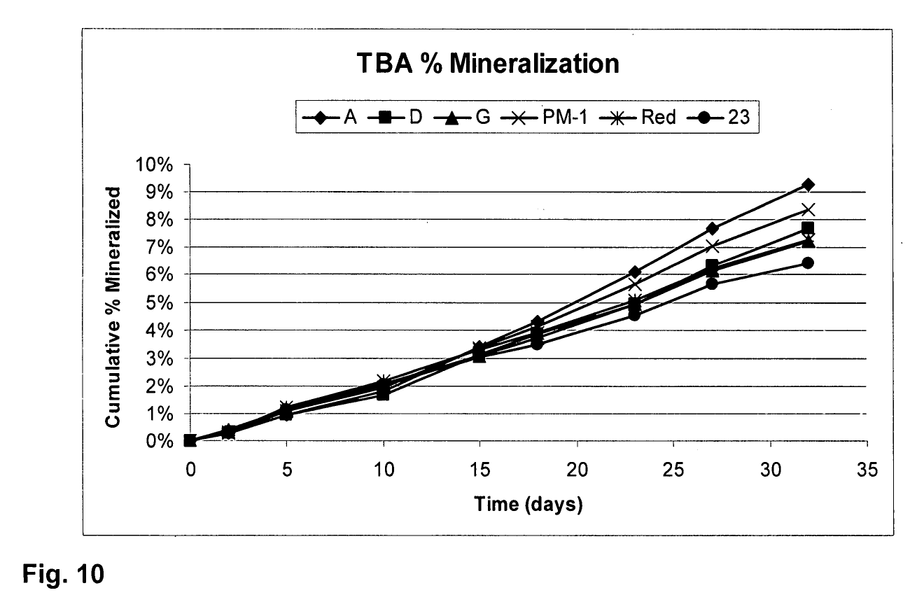

FIG. 10 is a graph of the ability of the M. KMS (shown as D), JLS (shown as A) and MCS (shown as G) strains to mineralize MTBE compared against mycobacterium PM-1, and two bacteria cultured from a Ronan site (shown as Red and 23). PM-1, Red and 23 indicated MTBE-degrading microorganisms used as positive controls. As shown, M. KMS was superior in degrading MTBE.

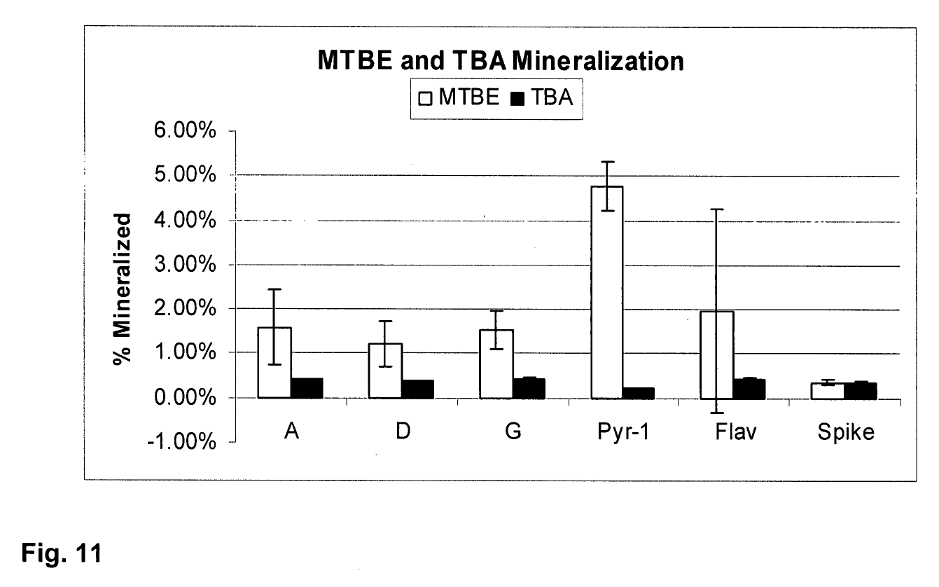

FIG. 11 is a graph of the ability of the M. KMS (shown as D), JLS (shown as A) and MCS (shown as G) strains to mineralize TBA compared against mycobacterium PM-1 (pos control), and two bacteria cultured from a Ronan site (shown as Red and 23). As shown, M. JLS was superior in degrading TBA.

### Example 13

The mycobacteria M. KMS, JLS, and MCS strains were tested for the ability to mineralize MTBE and tertbutyl acetate (“TBA”) in water, and compared against the mineralization ability of mycobacterium M. flavescens, and M. vanbalenni. Microcosms were incubated statically in the dark at 32° C. for optimum temperature. Controls included one pure culture not spiked with MTBE or TBA, and a culture with a distilled water spike. Concentrations of MTBE and TBA were set at 5 mg/L, and traps were sampled after 4 months.

FIG. 12 is a graph of the ability of the M. KMS (shown as D), JLS (shown as A) and MCS (shown as G) strains to mineralize MTBE and TBA in water compared against M. flavescens (“flav”), and M. vanbalenni (“PYR-1”). As shown, all of the mycobacteria were capable of enhanced MTBE mineralization compared to TBA mineralization. Additionally, flav and PYR-1 had higher mineralization for MTBE compared to M. KMS, JLS, and MCS.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A contaminant-degrading composition for use in remediation of contaminated soil having a selected contaminant, the composition comprising:
a seed for a plant capable of growing in the presence of the selected contaminant; and
a contaminant-degrading mycobacterium on the seed, the mycobacterium being capable of degrading the selected contaminant.

2. A composition as in claim 1, wherein the contaminant-degrading mycobacterium is at least one of M. KMS, M. JLS, or M. MCS.

3. A composition as in claim 1, wherein the contaminant-degrading mycobacterium has a nid dioxygenase gene.

4. A composition as in claim 3, wherein the nid dioxygenase gene includes a nidB-nidA sequence motif.

5. A composition as in claim 4, wherein the contaminant-degrading mycobacterium is capable of degrading a polycyclic aromatic hydrocarbon.

6. A composition as in claim 5, wherein the seed is for a plant selected from the group consisting of barley, wheatgrass, Lolium species, legumes, alfalfa, rice, grasses, forbs, trees, mulberry tree, clover, corn, brassicas, cucurbits and rye.

7. A composition as in claim 2, further comprising at least one of root wash, root extract, D-mannitol, D-psicose, propionic acid, D-sorbitol, sucrose, alpha-cyclodextrin, and sedoheptulosan, polyoxyethylene sorbitan mono-palmitate (Tween 40), polyoxyethylene sorbitan monooleate (Tween 80), D-fructose, D-mannose, D-trehalose, or pyruvic acid methyl ester.

8. A contaminant-degrading system for use in remediation of contaminated soil having a selected contaminant, the system comprising:
a plant having a root growing in the contaminated soil; and
contaminant-degrading mycobacteria colonized on the root of the plant, the mycobacteria being capable of degrading the selected contaminant.

9. A system as in claim 8, wherein the contaminant-degrading mycobacteria is at least one of M. KMS, M. JLS, or M. MCS.

10. A system as in claim 8, wherein the contaminant-degrading mycobacteria has a nid dioxygenase gene having a nidB-nidA sequence motif.

11. A system as in claim 10, wherein the contaminant-degrading mycobacteria is capable of degrading a polyaromatic hydrocarbon.

12. A system as in claim 11, wherein the plant selected from the group consisting of barley, wheatgrass, Lolium species, legumes, alfalfa, rice, grasses, forbs, trees, mulberry tree, clover, corn, brassicas, cucurbits, and rye.

13. A method of decontaminating soil having a selected contaminant, the method comprising:
growing a plant having a root in contaminated soil having a selected contaminant; and
colonizing contaminant-degrading mycobacteria on the root of the plant, the mycobacteria being capable of degrading the selected contaminant.

14. A method as in claim 13, further comprising planting a seed in the soil, said seed for a plant capable of growing in the presence of the selected contaminant.

15. A method as in claim 14, further comprising applying the contaminant-degrading mycobacteria to the seed.

16. A method as in claim 15, wherein the seed includes the contaminant-degrading mycobacteria before being planted.

17. A method as in claim 15, wherein the contaminant-degrading mycobacteria is applied to soil adjacent to at least one of the seed after planting or the plant.

18. A method as in claim 16, further comprising applying a composition having the contaminant-degrading mycobacteria to the soil.

19. A method as in claim 15, wherein the seed includes a liquid containing the contaminant-degrading mycobacteria at the time of planting.

20. A method as in claim 13, wherein the contaminant-degrading mycobacteria is at least one of M. KMS, M. JLS, or M. MCS.

21. A system as in claim 13, wherein the contaminant-degrading mycobacteria has a nid dioxygenase gene having a nidB-nidA sequence motif.
